(19) United States
(12) Patent Application Publication
(10) Pub. No.: US 2018/0127810 A1
(43) Pub. Date: May 10, 2018

(54) METHOD OF SEQUENCE AMPLIFICATION OF BACTERIAL STRAINS FOR BIOLOGICAL CONTROL AGAINST MOSQUITOES

(71) Applicant: YEDITEPE UNIVERSITESI, Istanbul (TR)

(72) Inventors: Fikrettin SAHIN, Istanbul (TR); Gulengul DUMAN, Istanbul (TR); Munevver Muge ÇAGAL, Bursa (TR)

(73) Assignee: YEDITEPE UNIVERSITESI, Istanbul (TR)

(21) Appl. No.: 15/791,387

(22) Filed: Oct. 23, 2017

Related U.S. Application Data
(63) Continuation-in-part of application No. 14/130,969, filed on Jan. 6, 2014, now abandoned.

(51) Int. Cl. C12Q 1/689; A01N 63/00

(52) U.S. Cl. CPC C12Q 1/689; C12Q 2600/156; A01N 63/00

(57) ABSTRACT

The present invention relates to novel bacteria strains that can be used in biological control against mosquito larvae (Cules spp.) and a method of sequence analysis for the strains. The sequence analysis method comprising genomic DNA extracting, 16S rDNA amplification, a PCR amplification of a 550 bp DNA segment and purifying and sequencing of the DNAs obtain via PCR. The protein obtained from a novel Bacillus sphaericus spp. Isolates with the invention is used as larvicide, the step of isolating the protein at product obtaining stage is eliminated. By means of the invention, the bacterial strains (MBI 5, 6, 7) investigated for biological control of mosquitoes are effective in both polluted and fresh water.

### CROSS REFERENCE TO RELATED APPLICATIONS

This application is continuation-in-part application of a U.S. application Ser. No. 14/130,969, filed on Jan. 6, 2014, the entire content of which is incorporated herein by reference.

### FIELD OF THE INVENTION

The present invention relates to a method of sequence analysis of novel bacteria strains in biological control against mosquito larvae (Culex spp.).

### BACKGROUND OF THE INVENTION

Mosquitoes are vectors of many diseases such as Mosquito-borne arboviruses, malaria, filariasis and Japan encephalitis. Generally, mosquito control is made by chemical pesticides more than bio-pesticides in the world. These chemical pesticides are known as dichlorodiphenyltrichloro ethane (DDT), gammaxane, malathion, chlordane and organophosphates. All of them have high toxic range for human health and environment. Compared to chemical pesticides, microbial insecticides are often species specific and do not contaminate environment, therefore, safe to non-target organisms in the nature. Among various microbial pesticides, Bacillus thrungiensis and Bacillus sphaericus are being widely used. Mosquitocidal bacteria are environmentally friendly alternatives to chemical pesticides for controlling water mosquitoes.

Bacillus thrungiensis subs. israilensis (Bti) is the most extensively used mosquito larvicidal bacteria in the world. Bti produces crystal glycoprotein (protoxin) coded by different genes such as Cry4A, Cry4B, Cry10A, Cry11A and Cry1A during sporulation. Bti Cry toxins have been widely used in the control of broad range of mosquito and blackfly species as well as nematodes, mite and protozoa. Another potential microbial pesticide, Bacillus sphaericus, is known to be effective against (Culex spp. and Anopheles spp. species, and has better residual activity in polluted waters by production of binary toxin (Bin) and mosquitocidal toxins (Mtx). Mosquito resistances to some of B. sphaericus strains carrying a single Bin (binary) toxin gene have been reported in many countries.

European Patent document no EP0349769, an application known in the state of the art, discloses Bacillus sphaericus bacteria genetically engineered with toxin producing genes taken from Bacillus thuringiensis var. israelensis (B.t.i.) bacteria and transferred to Bacillus sphaericus strains. The genetically modified (GM) Bacillus sphaericus strains produced are capable of producing B.t.i. toxins in effective amounts and can control against mosquito larvae and black flies effectively.

European Patent document no EP0454485. an application known in the state of the art, discloses using insect killing toxins obtained from Bacillus thuringiensis or Bacillus sphaericus bacteria against pests living in water such as mosquito larvae. The spores of these bacteria kill some insect larvae feeding on these spores. The spores are digested in intestines of the larvae and release their toxins and neutralize the larvae. The known applications in the technique disclose taking toxins of the bacteria to apply on larvae for biological control against mosquito. Taking the toxins of the bacteria requires extra labor and cost. That is to say, a protein isolation step is performed in these applications.

Many commercial products are introduced to the market, the resistance of mosquito populations to some known biological control products forces the scientists to search for new mosquitocidal bacterial strains which can be used for development of new commercial microbial insecticide.

### SUMMARY OF THE INVENTION

The objective of the present invention is to provide novel bacterial strains that can be used as larvicide in biological control.

A further objective of the present invention is to provide a sequence analysis method for the novel bacterial strains for biological control of mosquitoes.

Another objective of the present invention is to provide novel bacterial strains for biological control, wherein the toxin protein isolation step in product obtaining state is eliminated.

Another objective of the present invention is to provide novel bacterial strains for biological control which are effective in both polluted and fresh water.

### BRIEF DESCRIPTION OF THE DRAWINGS

FIG. 1: Bin 51 and bin 42 toxin genes PCR amplified by primers B.sph: B. sphaericus, MBI 5, MBI 6, and MBI 7.

FIG. 2: Mtx 1 and Mtx 2 toxin genes PCR amplified by primers B.sph: B. sphaericus, MBI 5, MBI 6, and MBI 7.

FIG. 3 is the PCR bands of MBI 5, MBI 6, and MBI 7 in gel imaging system (BIORAD) after electrophoresis in 1% agarose gel with ethidium bromide (NC: Negative Control).

FIG. 4 is a Neighbor-joining tree: the phylogenetic relationships among the Bacillus sphaericus-like strains.

FIG. 5 is the scanning electron microscope image of B. sphaericus bacterial cells.

FIG. 6 is the scanning electron microscope image of MBI5 bacterial strains.

FIG. 7 is the scanning electron microscope image of MBI6 bacterial strains.

FIG. 8 is the scanning electron microscope image of MBI7 bacterial strains.

FIG. 9 is the 16S rDNA sequence of MBI5 bacterial strain (SEQ ID NO:5).

FIG. 10 is the 16S rDNA sequence of MBI6 bacterial strain (SEQ ID NO:6).

FIG. 11 is the 16S rDNA sequence of MBI7 bacterial strain (SEQ ID NO:7).

### DETAILED DESCRIPTION OF THE INVENTION

in the inventive biological control against the mosquito larvae, the strains of B. sphaericus species are applied against the mosquito larvae. Deposit number is taken for the inventive strains from United States Department of Agriculture Research, Education and Economics Agricultural Research Service on Jan. 28, 2009. The deposit numbers of sub strains belonging to B. sphaericus species and named MBI 5, MBI 6, and MBI 7 are respectively registered as NRRL B-50199, NRRL B-50200 and NRRL B-50201.

In laboratory experiments carried out against mosquito larvae (Culex spp.), it has been found out that the larvicide effects and presence of Bin genes of B. sphaericus MBI 5, MBI 6, and MBI 7 strains are of the same as in the commercial strains of B. sphaericus. Furthermore, it has been observed that in experiments against the mosquito larvae the inventive bacteria strains show faster effect in a higher ratio than the known B. sphaericus strains. In the applications of the previous technique, an extra process is performed in order to obtain protein from the isolates. By means of the invention, the protein isolation step in obtaining product stage is eliminated. It is observed that newly found bacteria strains (MBI 5, MBI 6, and MBI 7) are effective when they are given to the medium in which the larvae present directly without performing protein isolation. At the same time Bacillus sphaericus strains show high larvicide effect both in polluted and fresh water. Various experimental studies have been carried out in order to test the effectiveness of the invention.

### Embodiment 1: Toxicity Test

Single colonies of newly isolated bacterial strains and Bti 4Q4, Bti ATCC 35646, B. sphaericus and were cultivated on to NYSM (Nutrient Yeast Salt Medium) agar and incubated for 48h at 30° C. Bacterial growth of each strain was harvested and resuspended in 10 ml of distilled water. Absorbance was adjusted to 0.2 with water and then 1 ml of suspension was added to 100 ml of fresh water/polluted water in 250 ml flasks containing 100 larvae (at the stage of 3 or 4^{th} instar) of Culex spp. The inoculated flasks were maintained on laboratory bench and observed for 48h at room temperature. In order to determine larvicidal bacterial strains, which were capable of killing 90% of larvae, positive and negative control flasks treated with reference strains and sterile water, respectively, were kept the same condition as inoculating ones. After toxicity test, three strains of B. sphaericus (MBI5, 6, 7) were selected as high toxic mosquitocidal bacteria and used for further studies. According to the bioassay test results, MBI 5, 6, 7 have a potential to be toxic to larvae of (Culex spp. Investigation of larvacidal features of three bacteria were done in fresh and polluted water that contained 100 larvae (see Table 1). Bti ATCC 35646, Bti 4Q4 and commercial B. sphaericus were used as positive control.

MBI 5 strain is isolated from diseased Culex spp. Larvae. MBI 6, and MBI 7 strains are isolated on a nutrient agar medium from diseased aphids.

TABLE 1
The effectiveness of MBI 5, MBI 6, and MBI 7 strains and
B. sphaericus, Bti ATCC 35646 and Bti 4Q4 bacteria against
Culex spp. larvae in polluted and fresh water.

| | Culex spp. Live larvae number | | |
|---|---|---|---|
| | (500 ml water/100 live larvae) | | |
| | Polluted water | | Fresh water |
| Bacteria name | 24 h | 48 h | 24 h |
| B. sphaericus (500 μL) | 10 | 4 | 0 |
| MBI 5 (500 μL) | 6 | 4 | 0 |
| MBI 6 (500 μL) | 7 | 3 | 0 |
| MBI 7 (500 μL) | 9 | 2 | 0 |
| BtiATCC35646 (500 μL) | 20 | 20 | 16 |
| Bti 4Q4 (500 μL) | 34 | 32 | 24 |

According to the test results, it was found that MBI 5, MBI 6, MBI 7 strains are more effective in polluted water within 24 hours compared to the known B. sphaericus, Bti ATCC 35646 and Bti 4Q4 bacteria. The effectiveness percentage of MBI 5, MBI 6, MBI 7 strains were determined as 94%, 93% and 91%, respectively. In tests performed in fresh water, it was observed that MBI 5, MBI 6, MBI 7 strains and B. sphaericus bacteria have 100% success rate by killing all existing healthy larvae within 24 hours. On the other hand, it was found out that Bti ATCC 35646 and Bti 4Q4 bacteria are effective against larvae at ratios of 84% and 76% in fresh water, respectively (Table 1).

### Embodiment 2: Phenotypic Diagnostic Studies

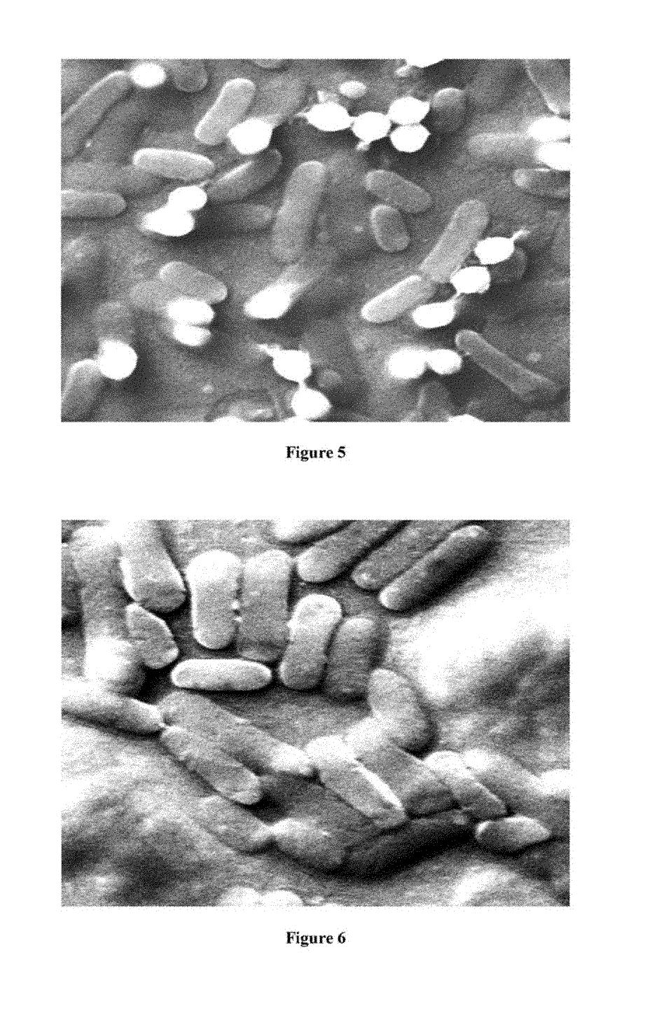

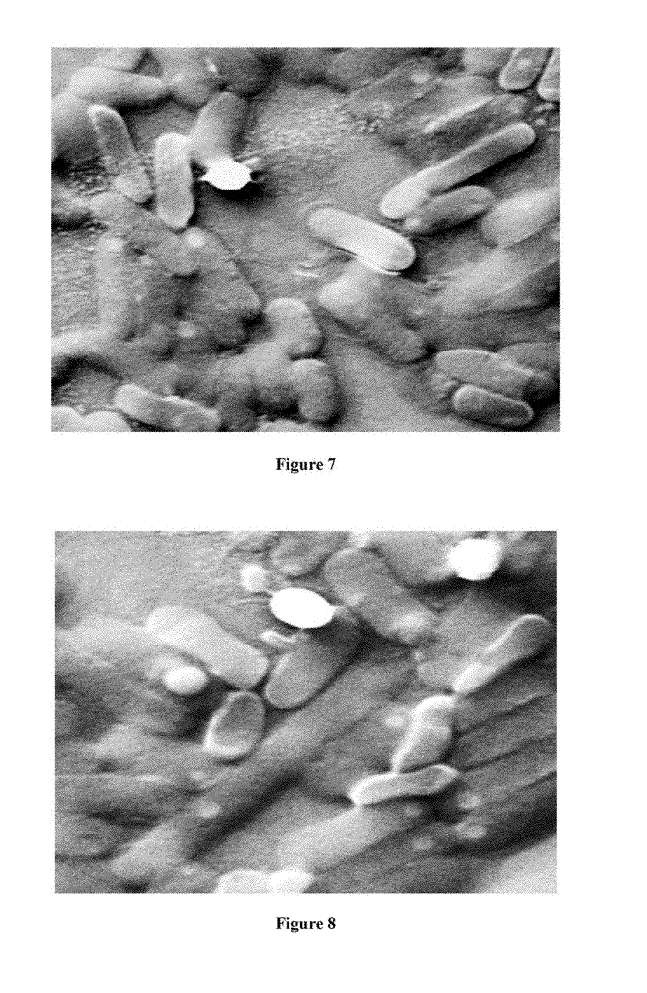

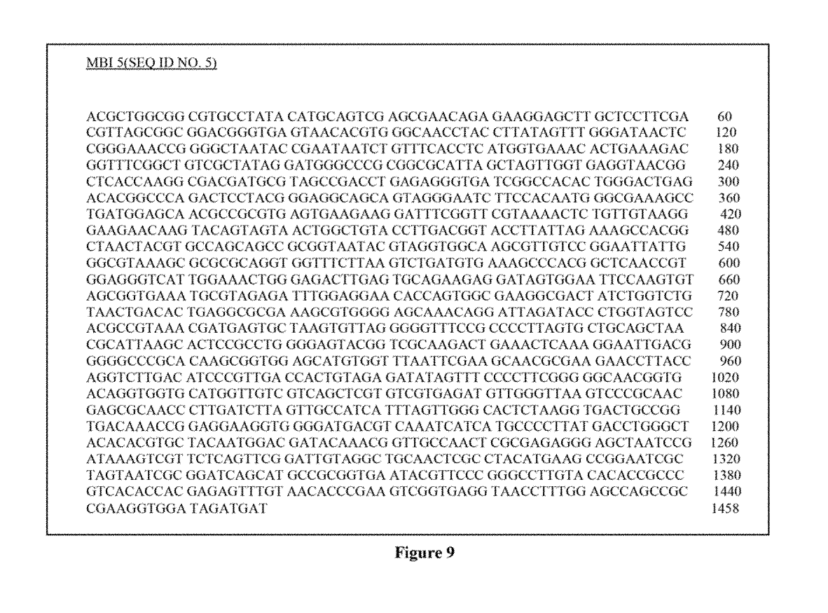

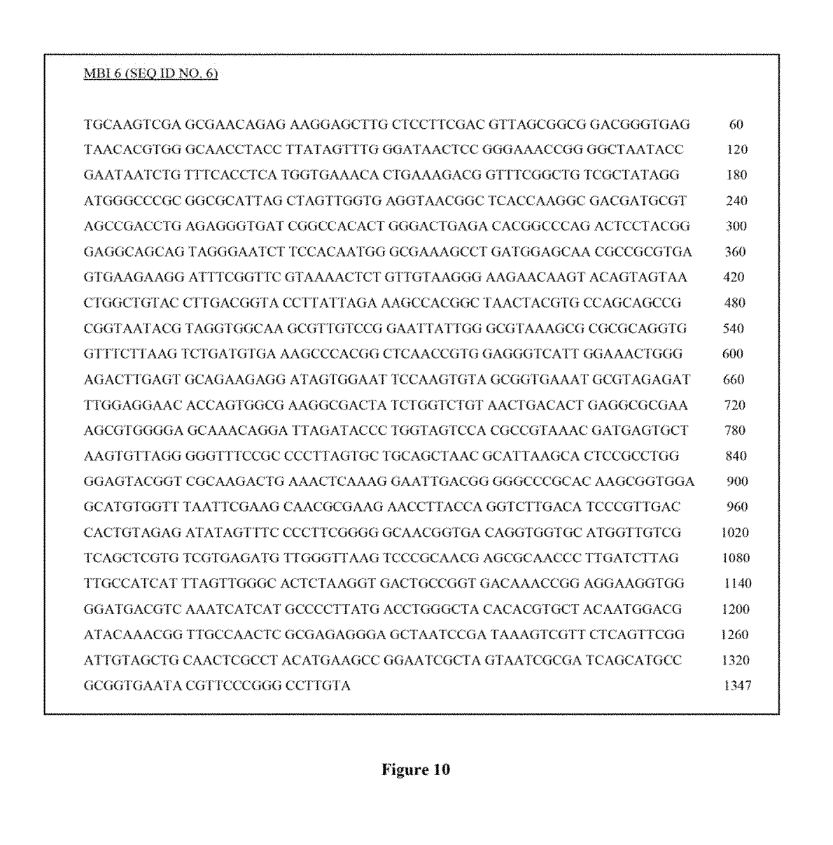

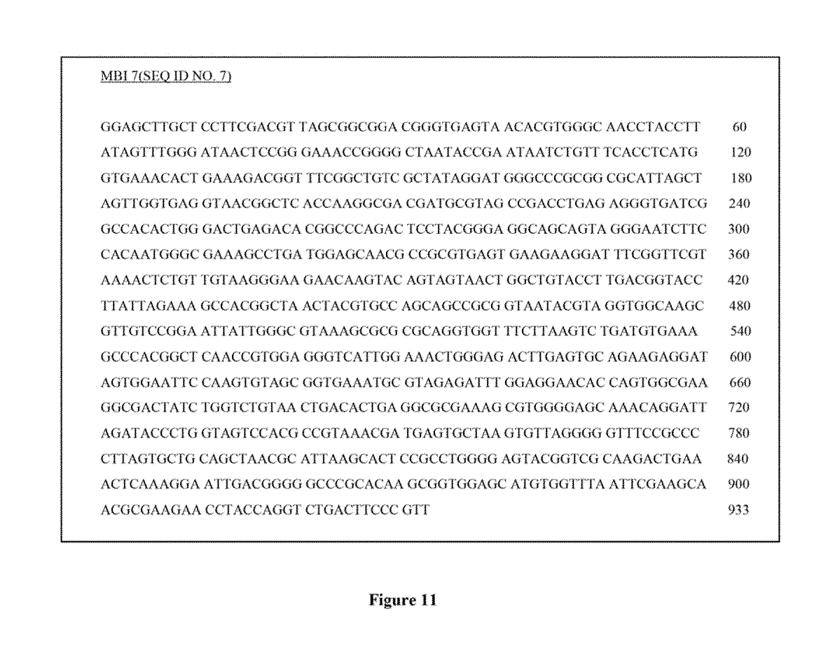

All of the methods provided to understand cell properties of three new strains of bacillus. MBI 5, MBI 6, MBI 7 were an aerobic, Gram-positive bacteria according to electron microscope images of MBI 5, MBI 6, MBI 7 and B. sphaericus, they are rod-shaped bacteria (FIG. 6, FIG. 7, and FIG. 8) and similar to B. sphaericus (FIG. 5).

They also grow at 20-35° C., and the optimum growth temperatures were 27-30° C. Growth at 50° C. and 4° C. were not observed on nutrient agar. The physiological characteristics of MBI 5, MBI 6, MBI 7 were summarized and selective characteristics with a related model such as B. sphaericus were compared (Table 2).

TABLE 2
Phenotypic characteristics of strains MBI5, MBI6, MBI7 compared with
commercial B. sphaericus. (+, positive; −, negative)

| Characteristics | MBI5 | MBI6 | MBI7 | B. sphaericus |
|---|---|---|---|---|
| Gram staining | + | + | + | + |
| Oxidase | − | − | − | − |
| Catalase | − | − | − | − |
| Capsule Staining | + | + | + | + |
| Endospor Staining | + | + | + | + |
| Hemolysis | + | + | + | + |
| Anaerobic test | − | − | − | − |
| Penicilline | + | + | + | + |

### Embodiment 3: Fatty Acid Profile Analysis

Each MBI strains were characterized as unique and novel in terms of BIOLOG, FAME profiles and 16S rRNA sequencing data.

The cellular fatty acid profiles of MBI 5. MBI 6, MBI 7 and B. sphaericus were listed in Table 3. The major cellular fatty acids in MBI 5 included iso-pentadecanoic acid (C_{15:0}, iso, 45.00%) and C_{16:0} iso, 12.65%. The minor fatty acid in MBI 5 included the iso-branched fatty acids C_{14:0} iso (0.60%), C_{16:0} (1.72%), C_{17:1} iso ω10c (1.43%). The major cellular fatty acids in MBI 6 included iso-pentadecanoic acid (C_{15:0} iso, 44.99%) and C_{16:0} iso, 15.24%. Minor amounts of the fatty acids C_{16:0} (0.78%), C_{17:1} iso ω10c (1.40%). The major cellular fatty acids in MBI 7 included iso-pentadecanoic acid (C_{15:0} iso, 45.84%) and C_{15:0} anteiso, 13,13%. Minor amounts of the iso-branched fatty acids C_{14:0} iso (0.68%), C_{18:1} iso ω9c (1.03%). Consequently, significant similarities in fatty acids profiles were found between B. sphaericus and MBI group. All of the groups MBI and B. sphaericus were identified with MIDI as Bacillus-sphaericus-GC subgroup E.

TABLE 3
Cellular fatty acid composition of MBI5, MBI6, MBI7 and B. sphaericus

| Numerical Names | | | | |
|---|---|---|---|---|
| of the Fatty acids | Percentage % | Percentage % | Percentage % | Percentage % |
| (Peak names) | MBI 5 | MBI 6 | MBI 7 | B. sphaericus |
| 14:0 iso | 2.02 | 4.38 | 1.51 | 1.26 |
| 14:0 | 0.60 | — | 0.68 | 0.85 |
| 15:0 iso | 45.00 | 44.99 | 45.84 | 46.61 |
| 15:0 anteiso | 10.87 | 9.22 | 13.13 | 7.89 |
| 14:0 iso 3OH | — | — | — | 1.05 |
| 16:1 w7c alkol | 9.93 | 12.38 | 9.55 | 6.80 |
| 16:iso | 12.65 | 15.24 | 8.14 | 5.48 |
| 16:1 w11c | 3.31 | 2.04 | 3.31 | 5.62 |
| 16:0 | 1.72 | 0.78 | 1.78 | 1.64 |
| 17:1 iso w10c | 1.43 | 1.40 | 2.35 | 4.92 |
| Sum In Feature 4 | 1.65 | 1.72 | 2.32 | 2.58 |
| 17:0 iso | 6.11 | 4.67 | 5.69 | 10.86 |
| 17:0 anteiso | 4.70 | 3.19 | 4.67 | 4.45 |
| 18:1 w9c | — | — | 1.03 | — |
| Summed Feature 4 | 1.65 | 1.72 | 2.32 | 2.58 |

### Embodiment 4: Sequence Analysis of 16S rDNA

DNA Extraction from Bacterial Strains:

Total genomic DNA from bacterial strains are extracted according to methodology described by Jimenez with some modifications. The pure strains are cultured in Nutrient Agar (NA) solid medium for 16-20 hours at 27° C. And one single colony is contaminated into 10 ml Nutrient Broth (NB) at 27° C. for 3-4 hours until the absorbances are up to 1 at 660 nm. The bacterial cells are collected from media after centrifugated for 10 min at 2000 g. The cells are suspended with 1 ml of Tris-EDTA buffer (10 mM Tris Base, 1 mM EDTA, 0.05% Tween 20, pH 9.0) and transferred into 2 ml micro-centrifuge tube, centrifuged at 14000 g for 2 min. Supernatant is discarded from the tube and 1 ml of Tris-EDTA buffer is added into the tube and repeat application for 3 times. Finally, 300 μl of Tris-EDTA buffer is added into the tube and boiled at 94° C. for 30 min in a water bath. After centrifuged at 14000 g for 2 min, 200 μl DNA is collected from supernatant and is stored at −20° C. for further PCR applications.

PCR Amplification and Purification of 16S rRNA:

16S rRNA genes of the bacterial DNA isolates (MBI5, MBI6, MBI7 and Bacillus sphaericus serotype H for control) are amplified by the PCR (BIORAD, Italy) using purified DNA and primers 27f and 1492r (Lane, 1991). The sequences of primers 27f (SEQ ID NO. 1) and 1492r (SEQ ID NO. 2) are shown as following:

27f (SEQ ID NO. 1): AGAGTTTGATCCTGGCTCAG;
1492r (SEQ ID NO. 2): CGGCTACCTTGTTACGAC.

PCR amplifications are carried out in a total volume of 50 ul reaction mixture containing 0.2 mM of 27f and 1492r primers respectively, 1 U of pfu DNA polymerase (Fermentas, USA), 0.2 mM of each deoxynucleoside triphosphate (dNTP), 1 mM of MgSO₄, 10 mM of Tris and 50 ng template DNA. PCR conditions are as follows: preamplification 94° C. for 5 min; denaturation at 94° C. for 30s, annealing at 55° C. for 40s, elongation at 72° C. for 2 min, repeat for 34 cycles: and then post amplification for a final extension of 10 min at 72° C.

Two new primers are designed for Bacillus sphaericus like members of Bacillaceae family. The new designed primers are FAM 1 (SEQ ID NO. 3) and FAM 2 (SEQ ID NO.4), the sequences of the primers are shown as following:

FAM 1 (SEQ ID NO. 3): CTCTGTTGTAAGGGAAGAAC;
FAM 2 (SEQ ID NO. 4): CCATGCACCACCTGTCACCG.

550 bp of 16S rRNA gene fragments of the bacterial DNA isolates (MBI5, MBI6, MBI7 and Bacillus sphaericus serotype H for control) are amplified by PCR (BIORAD, Italy) using purified DNA and primers FAM1 and FAM2. PCR amplifications are carried out in a total volume of 50 ul reaction mixture containing 0.2 mM of FAM1 and FAM2 primers respectively for a 550 bp DNA segment of 16S rDNA, 1 U of pfu DNA polymerase (Fermentas, USA), 0.2 mM of each deoxynucleoside triphosphate (dNTP), 1 mM of MgSO₄, 10 mM of Tris and 50 ng template DNA. PCR conditions are as follows: preamplification 94° C. for 5 min; denaturation at 94° C. for 30s, annealing at 51° C. for 40s, elongation at 72° C. for 45 sec, repeat for 34 cycles; and then post amplification for final extension of 10 min at 72° C.

The amplified DNA products are detected by Biorad image analysing system (BIORAD, Italy) after electrophoresis of PCR amplicons in a 1% agarose gel stained with ethidium bromide.

16S rRNA Gene Sequencing and Phylogenetic Analysis

Pure amplification products are sequenced with a Prism ABI 3100 Genetic Analyzer 16 caillaries. dideoxy terminator cycle sequencing kit (Applied Biosystems). The protocols used are due to manufacturers recommendations. Sequences are determined with an automated DNA sequencer (model: Prism ABI 3100; Applied Biosystems). Both strands are sequenced using the primers 27f, 1492r. FAM1 and FAM2 (Lane, 1991; Nakamura, 1996). The clustal w program (Higgins et al., 1992) is used to align the 16S DNA sequences generated with sequences of Bacillus sphaericus like members from GenBank NCBI (Larsen et al., 1993). The sequences of 16s rDNA genes are obtained. The 16S rDNA sequence of MBI 5 is shown in SEQ ID NO. 5. The 16S rDNA sequence of MBI 6 is shown in SEQ ID NO. 6. And the 16S rDNA sequence of MBI 7 is shown in SEQ ID NO. 7.

Genetic distance is computed using Kimura's two-parameter model (Kimura, 1980) and used for neighbor-joining analysis. Phylogenetic trees are constructed using neighbor-joining and maximum-parsimony methods provided by CLC Genomics Workbench_2_1_1 both methods produce trees with similar topologies. Nucleotide sequences generated in this study have been deposited with GenBank under the accession numbers.

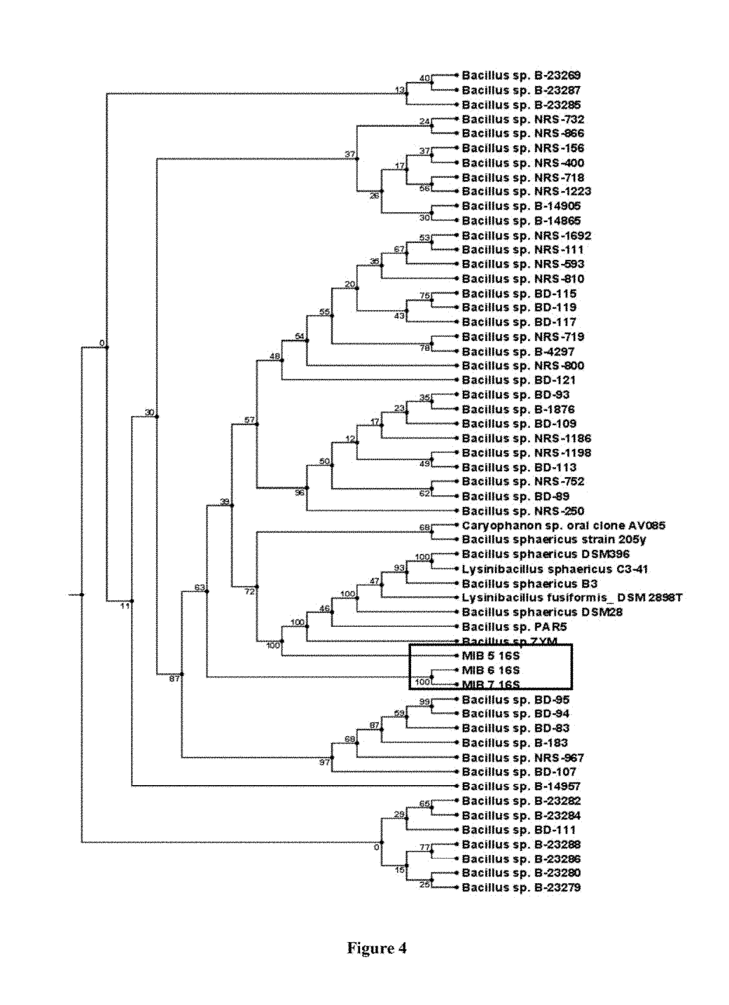

As shown in FIG. 4, another study is neighbor-joining tree analysis that is based on 1450 nucleotide sequences. Confidence limits estimated from bootstrap analyses (100 replications) appear at the nodes. A maximum-parsimony tree generated from the sequence data exhibit similar topology to this tree. In the phylogenetic tree; MBI 5, MBI 6, MBI 7 clearly belong to the strains of Bacillus sphaericus, as shown by the high bootstrap value (FIG. 4).

As shown in FIG. 4, MBI 5 (SEQ ID NO. 5). MBI 6 (SEQ ID NO. 6), and MBI 7 (SEQ ID NO. 7) strains are located between Bacillus sp. ZYM and Bacillus sp. BD-95. Further in view of 16S rDNA sequences, MBI 5, MBI 6, MBI 7 share a high level of gene sequence similarity with Bacillus sp. ZYM and Bacillus sp. BD-95. However, no matter how close they are, MBI 5 (SEQ ID NO. 5), MBI 6 (SEQ ID NO. 6), MBI 7 (SEQ ID NO. 7), Bacillus sp. ZYM and Bacillus sp. BD-95 are still different sub-species, so they do not have 100% homology.

### Embodiment 5: Determination of Toxin Genes

Toxin genes are investigated according to methodology described by Nishiwaki et al., PCR of toxin genes of the bacterial DNA isolates (MBI 5 (SEQ ID NO. 5), MBI 6 (SEQ ID NO. 6), and MBI 7 (SEQ ID NO. 7) and Bacillus sphaericus serotype H for control) is done for the genes encoding the mosquitocidal binary toxin (51 and 42 kDa), Mtx1, and Mtx2. PCR is constructed according to the following conditions: preamplification 94° C. for 2 min followed by 30 cycles of denaturation at 94° C. for 15 s, annealing at 55° C. for 30 s, and elongation at 72° C. for 1 min 30 s. The master mix consists of 1 U of TSG polymerase (Biobasic, Canada), 1 mM of MgSO₄, 0.2 mM of each deoxynucleoside triphosphate (dNTP), 20 ng of template DNA, and 5 pmol of each primer in total volume of 50 ul reaction mixture.

TABLE 4
Primers used in determination
of toxin genes

| Sequence ID | Primer | |
|---|---|---|
| Number | name | Sequence |
| SEQ ID NO. 8 | 51 kDa F | CGCTAAATACTACTCCTACAAGCC |
| SEQ ID NO. 9 | 51 kDa R | GGATACGATTGTATATACCTGCC |
| SEQ ID NO. 10 | 42 kDa F | CCCACAGAAGGAAAGTACATTCGC |
| SEQ ID NO. 11 | 42 kDa R | CCTAGTAAAGGTTCACTTGC |
| SEQ ID NO. 12 | Mtx1 F | CAAGCTGCTTCACTTACATG |
| SEQ ID NO. 13 | Mtxl R | GTCCAGTTACATCTTGAGCC |
| SEQ ID NO. 14 | Mtx2 F | GGAGACTAATTGAATTTTCGGTTTCC |
| SEQ ID NO. 15 | Mtx2 R | GCGATGCTGGGCTATGTTCGTTCGTTA |

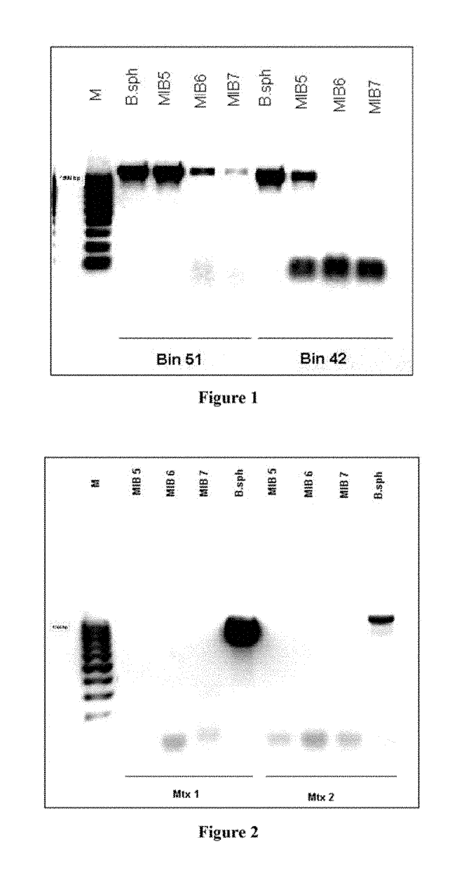

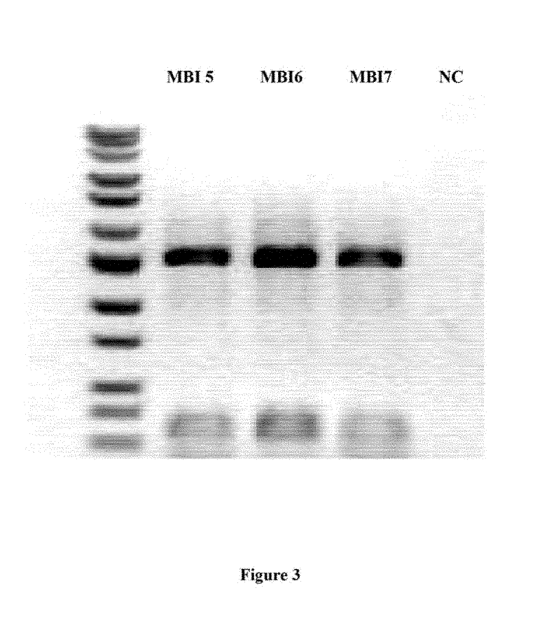

The amplified DNA products was detected by using Biorad image analyzing system (BIORAD, Italy) after electrophoresis of PCR amplicons in a 1% agarose gel stained with ethidium bromide (FIG. 1, FIG. 2).

The PCR amplification of Bin and Mtx toxin genes of MBI 5, MBI 6, MBI 7 and commercial B. Sphaericus have been done. FIG. 1 reveals that B. sphaericus, MBI 5, MBI 6, MBI 7 have Bin 51 and Bin 42 toxin genes. At the same time, MBI 5, MBI 6, MBI 7 have no Mtx1 and Mtx 2 toxin genes (FIG. 2). In addition, commercial B. sphaericus has both Bin and Mtx toxins.

What is claimed is:

1. A method of sequence analysis of bacterial strains for biological control against mosquitoes, comprising the following steps:
(1) culturing a pure strain;
(2) total genomic DNA extracting;
(3) 16S rDNA amplification: amplifying using primers of 27f and 1492r;
(4) PCR amplification of a 550 bp DNA segment of the 16S rDNA: amplifying using primers of FAM 1 and FAM 2; wherein FAM 1 having a sequence shown in SEQ ID NO. 3, FAM 2 having a sequence shown in SEQ ID NO. 4;
(5) purifying and sequencing both the 16S rDNA and the 550 bp DNA segment of the 16S rDNA.

2. The method according to claim 1, wherein the 16S rDNA PCR amplification is carried out in a reaction mixture with a total volume of 50 μl comprising 0.2 mM of each primer respectively, 1 U of pfu DNA polymerase, 0.2 mM of each deoxynucleoside triphosphate (dNTP), 1 mM of MgSO₄, 10 mM of Tris and 50 ng template DNA; wherein a condition for the PCR amplification is: preamplification at 94° C. for 5 min followed by 34 cycles of denaturation at 94° C. for 30s, annealing at 55° C. for 40s, elongation at 72° C. for 2 min; and then post amplification for a final extension of 10 min at 72° C.

3. The method according to claim 1, wherein the PCR amplification of the 550 bp DNA segment of the 16S rDNA is carried out in a reaction mixture with a total volume of 50 μl comprising 0.2 mM of each primer respectively, 1 U of pfu DNA polymerase, 0.2 mM of each deoxynucleoside triphosphate (dNTP), 1 mM of MgSO₄, 10 mM of Tris and 50 ng template DNA; wherein a condition for the PCR amplification is: preamplification at 94° C. for 5 min followed by 34 cycles of denaturation at 94° C. for 30s, annealing at 51° C. for 40s, elongation at 72° C. for 45s; and then post amplification for a final extension of 10 min at 72° C. to obtain a 550 bp DNA segment of the 16S rDNA.

4. The method according to claim 1, wherein the culturing of the pure strain comprises steps of culturing the strain in a Nutrient Agar solid medium at 27° C. for 16-20 h and contaminating a single colony of the pure strain into a Nutrient Broth at 27° C. for 3-4 h to obtain a culture solution with an absorbance up to 1 at 660 nm.

5. The method according to claim 1, wherein the total genomic DNA extracting comprises steps of collecting cells of the strain from a culture solution by centrifugation at 2000 g for 10 min; suspending the cells with 1 mL of Tris-EDTA buffer, transferring the cells into a micro-centrifuge tube, centrifuging at 140000 g for 2 min, discarding the supernatant obtained after the centrifugation, adding 1 mL of Tris-EDTA buffer into the tube and repeat the centrifuging and discarding of the supernatant obtained after the centrifugation for 3 times; adding 300 μl of Tris-EDTA buffer into the tube to obtain a solution of cells, boiling the solution of cells at 94° C. for 30 min, centrifuging at 14000 g for 2 min, collecting 200 μl supernatant of the boiled solution to obtain 200 μl DNA.

6. The method according to claim 1, wherein the strains for biological control against mosquitoes comprises strains of MBI 5, MBI 6 and MBI 7 belonging to Bacillus sphaericus species registered as NRRL B-50199, NRRL B-50200 and NRRL B-50201 respectively, wherein the deposit numbers are taken from United States Department of Agriculture Research, Education and Economics Agricultural Research Service on Jan. 28, 2009.


SEQUENCE LISTING (15 sequences)

SEQ ID NO: 1 - DNA, 20 bp, Artificial Sequence
  The sequence is synthesized.
agagtttgatcctggctcag

SEQ ID NO: 2 - DNA, 18 bp, Artificial Sequence
  The sequence is synthesized.
cggctaccttgttacgac

SEQ ID NO: 3 - DNA, 20 bp, Artificial Sequence
  The sequence is synthesized.
ctctgttgtaagggaagaac

SEQ ID NO: 4 - DNA, 20 bp, Artificial Sequence
  The sequence is synthesized.
ccatgcaccacctgtcaccg

SEQ ID NO: 5 - DNA, 1458 bp, Bacillus sphaericus
acgctggcggcgtgcctatacatgcagtcgagcgaacagagaaggagcttgctccttcgacgttagcggcggacgggtgagtaacacgtgggcaacctaccttatagtttgggataactccgggaaaccggggctaataccgaataatctgtttcacctcatggtgaaacactgaaagacggtttcggctgtcgctataggatgggcccgcggcgcattagctagttggtgaggtaacggctcaccaaggcgacgatgcgtagccgacctgagagggtgatcggccacactgggactgagacacggcccagactcctacgggaggcagcagtagggaatcttccacaatgggcgaaagcctgatggagcaacgccgcgtgagtgaagaaggatttcggttcgtaaaactctgttgtaagggaagaacaagtacagtagtaactggctgtaccttgacggtaccttattagaaagccacggctaactacgtgccagcagccgcggtaatacgtaggtggcaagcgttgtccggaattattgggcgtaaagcgcgcgcaggtggtttcttaagtctgatgtgaaagcccacggctcaaccgtggagggtcattggaaactgggagacttgagtgcagaagaggatagtggaattccaagtgtagcggtgaaatgcgtagagatttggaggaacaccagtggcgaaggcgactatctggtctgtaactgacactgaggcgcgaaagcgtggggagcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccgttgaccactgtagagatatagtttccccttcgggggcaacggtgacaggtggtgcatggttgtcgtcagctcgtgtcgtgagatgttgggttaagtcccgcaacgagcgcaacccttgatcttagttgccatcatttagttgggcactctaaggtgactgccggtgacaaaccggaggaaggtggggatgacgtcaaatcatcatgccccttatgacctgggctacacacgtgctacaatggacgatacaaacggttgccaactcgcgagagggagctaatccgataaagtcgttctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtgaggtaacctttggagccagccgccgaaggtggatagatgat

SEQ ID NO: 6 - DNA, 1347 bp, Bacillus sphaericus
tgcaagtcgagcgaacagagaaggagcttgctccttcgacgttagcggcggacgggtgagtaacacgtgggcaacctaccttatagtttgggataactccgggaaaccggggctaataccgaataatctgtttcacctcatggtgaaacactgaaagacggtttcggctgtcgctataggatgggcccgcggcgcattagctagttggtgaggtaacggctcaccaaggcgacgatgcgtagccgacctgagagggtgatcggccacactgggactgagacacggcccagactcctacgggaggcagcagtagggaatcttccacaatgggcgaaagcctgatggagcaacgccgcgtgagtgaagaaggatttcggttcgtaaaactctgttgtaagggaagaacaagtacagtagtaactggctgtaccttgacggtaccttattagaaagccacggctaactacgtgccagcagccgcggtaatacgtaggtggcaagcgttgtccggaattattgggcgtaaagcgcgcgcaggtggtttcttaagtctgatgtgaaagcccacggctcaaccgtggagggtcattggaaactgggagacttgagtgcagaagaggatagtggaattccaagtgtagcggtgaaatgcgtagagatttggaggaacaccagtggcgaaggcgactatctggtctgtaactgacactgaggcgcgaaagcgtggggagcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccgttgaccactgtagagatatagtttccccttcgggggcaacggtgacaggtggtgcatggttgtcgtcagctcgtgtcgtgagatgttgggttaagtcccgcaacgagcgcaacccttgatcttagttgccatcatttagttgggcactctaaggtgactgccggtgacaaaccggaggaaggtggggatgacgtcaaatcatcatgccccttatgacctgggctacacacgtgctacaatggacgatacaaacggttgccaactcgcgagagggagctaatccgataaagtcgttctcagttcggattgtagctgcaactcgcctacatgaagccggaatcgctagtaatcgcgatcagcatgccgcggtgaatacgttcccgggccttgta

SEQ ID NO: 7 - DNA, 933 bp, Bacillus sphaericus
ggagcttgctccttcgacgttagcggcggacgggtgagtaacacgtgggcaacctaccttatagtttgggataactccgggaaaccggggctaataccgaataatctgtttcacctcatggtgaaacactgaaagacggtttcggctgtcgctataggatgggcccgcggcgcattagctagttggtgaggtaacggctcaccaaggcgacgatgcgtagccgacctgagagggtgatcggccacactgggactgagacacggcccagactcctacgggaggcagcagtagggaatcttccacaatgggcgaaagcctgatggagcaacgccgcgtgagtgaagaaggatttcggttcgtaaaactctgttgtaagggaagaacaagtacagtagtaactggctgtaccttgacggtaccttattagaaagccacggctaactacgtgccagcagccgcggtaatacgtaggtggcaagcgttgtccggaattattgggcgtaaagcgcgcgcaggtggtttcttaagtctgatgtgaaagcccacggctcaaccgtggagggtcattggaaactgggagacttgagtgcagaagaggatagtggaattccaagtgtagcggtgaaatgcgtagagatttggaggaacaccagtggcgaaggcgactatctggtctgtaactgacactgaggcgcgaaagcgtggggagcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaacctaccaggtctgacttcccgtt

SEQ ID NO: 8 - DNA, 24 bp, Artificial Sequence
  The sequence is synthesized.
cgctaaatactactcctacaagcc

SEQ ID NO: 9 - DNA, 21 bp, Artificial Sequence
  The sequence is synthesized.
ggatacgattgtatacctgcc

SEQ ID NO: 10 - DNA, 24 bp, Artificial Sequence
  The sequence is synthesized.
cccacagaaggaaagtacattcgc

SEQ ID NO: 11 - DNA, 20 bp, Artificial Sequence
  The sequence is synthesized.
cctagtaaaggttcacttgc

SEQ ID NO: 12 - DNA, 20 bp, Artificial Sequence
  The sequence is synthesized.
caagctgcttcacttacatg

SEQ ID NO: 13 - DNA, 20 bp, Artificial Sequence
  The sequence is synthesized.
gtccagttacatcttgagcc

SEQ ID NO: 14 - DNA, 26 bp, Artificial Sequence
  The sequence is synthesized.
ggagactaattgaattttcggtttcc

SEQ ID NO: 15 - DNA, 26 bp, Artificial Sequence
  The sequence is synthesized.
gcgatgctgggctatgttcgttgtta